(19) United States
(12) Patent Application Publication
(10) Pub. No.: US 2003/0143185 A1
(43) Pub. Date: Jul. 31, 2003

(54) Polymer conjugates of protein kinase C inhibitors

(75) Inventors: Michael David Bentley, Huntsville, AL (US); Xuan Zhao, Huntsville, AL (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Shearwater Corporation

(21) Appl. No.: 10/282,915

(22) Filed: Oct. 29, 2002

Related U.S. Application Data
(60) Provisional application No. 60/340,535, filed on Oct. 29, 2001.

(51) Int. Cl. A61K 31/785; A61K 31/7052; A61K 38/16; C07K 14/00; C08G 69/00

(52) U.S. Cl. 424/78.17; 514/2; 514/54; 536/17.4; 530/409; 525/204; 525/435

(57) ABSTRACT

The invention provides polymer conjugates of protein kinase C (PKC) inhibitors comprising a polymer, such as poly(ethylene glycol), covalently attached to a PKC inhibitor, such as a bisindolylmaleimide molecule. The linkage between the polymer and the PKC inhibitor is preferably hydrolytically degradable. The invention also includes a pharmaceutical composition comprising a polymer conjugate of a PKC inhibitor and a method of treating any condition responsive to a PKC inhibitor by administering a polymer conjugate of the invention.

### CROSS-REFERENCE TO RELATED APPLICATIONS

This application claims the benefit of priority of Provisional Application Serial No. 60/340,535, filed Oct. 29, 2001, which is incorporated herein by reference in its entirety.

### FIELD OF THE INVENTION

This invention relates to water-soluble polymer conjugates of biologically active molecules, and in particular, to water-soluble polymer conjugates of protein kinase C inhibitors, and related pharmaceutical compositions and uses thereof.

### BACKGROUND OF THE INVENTION

Bisindolylmaleimides are a subgroup of a larger family of natural products known as indolocarbazoles. Many members of the indolocarbazole family have shown activity as antimicrobial, antifungal, immunosuppressive, and antitumor agents, as well as protein kinase inhibitors.

Much of the indolocarbazole research has focused on the promising role of many bisindolylmaleimide compounds as selective protein kinase C (PKC) inhibitors. Due to the pivotal role that the PKC enzyme plays in cell-cell signaling, gene expression, and in the control of cell differentiation and growth, it is implicated in the pathogenesis of a variety of diseases, including cancer, autoimmune diseases such as rheumatoid arthritis, hypertension, and asthma. Protein kinase C is composed of twelve isozymes: alpha (α), beta-I (β-I), beta-II (β-II), gamma (γ), delta (δ), epsilon (ε), eta (η), theta (θ), mu (μ), zeta (ξ), lambda (λ), and iota (ι). Since PKC may exist as many different isozymes, only one or two of which may be involved in a given disease state, there remains a need for therapeutically effective isozyme-selective inhibitors. Accordingly, several bisindolylmaleimide compounds have been identified as potent and selective PKC inhibitors. See, Davis et al., FEBS Lett. 259(1):61-63 (1989); Twomey et al., Biochem. Biophys. Res. Commun. 171(3):1087-1092 (1990); Toullec, et al., J. Biol. Chem. 266(24): 15771-15781 (1991); Davis et al., J. Med. Chem. 35:994-1001 (1992); Bit et al., J. Med. Chem. 36:21-29 (1993); WO 99/44606; EP 0 940 141 A2; WO 99/44607.

Although bisindolylmaleimides having therapeutic activity are known, it has been noted that effective kinase inhibitors should be capable of rapidly crossing cell membranes and, ideally, possess oral activity in mammals. See Bishop et al., TRENDS in Cell Biology 11(4) 167-172 (2001). However, poor oral bioavailability due to low aqueous solubility has limited the therapeutic utility of many bisindolylmaleimides. Thus, there is a need in the art for alternative compounds, or for approaches for modifying or improving upon existing compounds, that can maintain at least a certain degree or enhance the therapeutic activity of bisindolylmaleimide compounds and other PKC inhibitors, while increasing solubility and bioavailability.

### SUMMARY OF THE INVENTION

The present invention is based upon the development of water-soluble, polymer-modified PKC inhibitors designed for the treatment of PKC mediated diseases. In one aspect, the present invention provides a polymer conjugate comprising a water-soluble and non-peptidic polymer covalently attached, preferably through a hydrolytically degradable linkage, to a PKC inhibitor molecule, such as a bisindolylmaleimide molecule.

Suitable polymers for covalent attachment to a PKC inhibitor include poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), poly(acrylic acid), carboxymethyl cellulose, hyaluronic acid, hydroxypropylmethyl cellulose, and copolymers, terpolymers, and mixtures thereof. In one embodiment of the invention, the polymer is a poly(ethylene glycol).

The polymer portion of a conjugate of the invention may be linear, such as methoxy PEG, branched, or forked. In particular embodiments of the invention wherein the polymer is linear, the conjugate may incorporate a heterobifunctional or a homobifunctional polymer. A conjugate of a heterobifunctional polymer is one wherein one terminus of the polymer is attached to the PKC inhibitor and the other terminus is functionalized with a different moiety. A conjugate of a homobifunctional polymer possesses a structure wherein each end of a linear polymer is covalently attached to a PKC inhibitor, typically by an identical linkage.

In another aspect, the invention encompasses a pharmaceutical composition comprising a polymer conjugate as described above in combination with a pharmaceutically acceptable carrier.

According to yet another aspect, the invention provides a method of treating any condition responsive to PKC inhibition, such as various inflammatory diseases and conditions, immunological diseases, bronchopulmonary diseases, cardiovascular diseases, diabetes, dermatological diseases, cancer, and central nervous system (CNS) diseases, by administering a polymer conjugate as described above.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

I. Definitions

The following terms as used herein have the meanings indicated.

As used in the specification, and in the appended claims, the singular forms “a”, “an”, “the”, include plural referents unless the context clearly dictates otherwise.

The terms “functional group”, “active moiety”, “reactive site”, “chemically reactive group” and “chemically reactive moiety” are used in the art and herein to refer to distinct, definable portions or units of a molecule. The terms are somewhat synonymous in the chemical arts and are used herein to indicate the portions of molecules that perform some function or activity and are reactive with other molecules. The term “active,” when used in conjunction with a functional group, is intended to include those functional groups that react readily with electrophilic or nucleophilic groups on other molecules, in contrast to those groups that require strong catalysts or highly impractical reaction conditions in order to react (i.e., “non-reactive” or “inert” groups). For example, as would be understood in the art, the term “active ester” would include those esters that react readily with nucleophilic groups such as amines. Exemplary active esters include N-hydroxysuccinimidyl esters or 1-benzotriazolyl esters. Typically, an active ester will react with an amine in aqueous medium in a matter of minutes, whereas certain esters, such as methyl or ethyl esters, require a strong catalyst in order to react with a nucleophilic group. As used herein, the term “functional group” includes protected functional groups.

The term “protected functional group” or “protecting group” or “protective group” refers to the presence of a moiety (i.e., the protecting group) that prevents or blocks reaction of a particular chemically reactive functional group in a molecule under certain reaction conditions. The protecting group will vary depending upon the type of chemically reactive group being protected as well as the reaction conditions to be employed and the presence of additional reactive or protecting groups in the molecule, if any. Protecting groups known in the art can be found in Greene, T. W., et al., PROTECTIVE GROUPS IN ORGANIC SYNTHESIS, 3rd ed., John Wiley & Sons, New York, N.Y. (1999).

The term “linkage” or “linker” (L) is used herein to refer to an atom or a collection of atoms used to link, preferably by one or more covalent bonds, interconnecting moieties such as two polymer segments or a terminus of a polymer and a reactive functional group present on a bioactive agent, such as a PKC inhibitor. A linker of the invention may be hydrolytically stable or may include a physiologically hydrolyzable or enzymatically degradable linkage.

A “physiologically hydrolyzable” or “hydrolytically degradable” bond is a weak bond that reacts with water (i.e., is hydrolyzed) under physiological conditions. Preferred are bonds that have a hydrolysis half life at pH 8, 25° C. of less than about 30 minutes. The tendency of a bond to hydrolyze in water will depend not only on the general type of linkage connecting two central atoms but also on the substituents attached to these central atoms. Appropriate hydrolytically unstable or degradable linkages include but are not limited to carboxylate ester, phosphate ester, anhydrides, acetals, ketals, acyloxyalkyl ether, imines, orthoesters, peptides and oligonucleotides.

A “hydrolytically stable” linkage or bond refers to a chemical bond, typically a covalent bond, that is substantially stable in water, that is to say, does not undergo hydrolysis under physiological conditions to any appreciable extent over an extended period of time. Examples of hydrolytically stable linkages include but are not limited to the following: carbon-carbon bonds (e.g., in aliphatic chains), ethers, amides, urethanes, and the like. Generally, a hydrolytically stable linkage is one that exhibits a rate of hydrolysis of less than about 1-2% per day under physiological conditions. Hydrolysis rates of representative chemical bonds can be found in most standard chemistry textbooks.

An “enzymatically unstable” or degradable linkage is a linkage that can be degraded by one or more enzymes.

The term “polymer backbone” refers to the covalently bonded chain of repeating monomer units that form the polymer. The terms polymer and polymer backbone are used herein interchangeably. For example, the polymer backbone of PEG is —CH₂CH₂O—(CH₂CH₂O)ₙ—CH₂CH₂where n typically ranges from about 2 to about 4000. As would be understood, the polymer backbone may be covalently attached to terminal functional groups or pendant functionalized side chains spaced along the polymer backbone.

The term “reactive polymer” refers to a polymer bearing at least one reactive functional group.

Unless otherwise noted, molecular weight is expressed herein as number average molecular weight (Mₙ), which is defined as

$$\frac{\sum NiMi}{\sum Ni} ,$$

wherein Ni is the number of polymer molecules (or the number of moles of those molecules) having molecular weight Mi.

The term “alkyl”, “alkenyl”, and “alkynyl” refers to hydrocarbon chains typically ranging from about 1 to about 12 carbon atoms in length, preferably 1 to about 6 atoms, and includes straight and branched chains. Unless otherwise noted, the preferred embodiment of any alkyl referred to herein is C1-C6alkyl (e.g., methyl or ethyl).

“Cycloalkyl” refers to a saturated or unsaturated cyclic hydrocarbon chain, including bridged, fused, or spiro cyclic compounds, preferably comprising 3 to about 12 carbon atoms, more preferably 3 to about 8.

The term “substituted alkyl”, “substituted alkenyl”, “substituted alkynyl” or “substituted cycloalkyl” refers to an alkyl, alkenyl, alkynyl or cycloalkyl group substituted with one or more non-interfering substituents, such as, but not limited to, C3-C8 cycloalkyl, e.g., cyclopropyl, cyclobutyl, and the like; acetylene; cyano; alkoxy, e.g., methoxy, ethoxy, and the like; lower alkanoyloxy, e.g., acetoxy; hydroxy; carboxyl; amino; lower alkylamino, e.g., methylamino; ketone; halo, e.g. chloro or bromo; phenyl; substituted phenyl, and the like.

“Alkoxy” refers to an —O—R group, wherein R is alkyl or substituted alkyl, preferably C1-C6 alkyl (e.g., methoxy or ethoxy).

“Aryl” means one or more aromatic rings, each of 5 or 6 core carbon atoms. Multiple aryl rings may be fused, as in naphthyl or unfused, as in biphenyl. Aryl rings may also be fused or unfused with one or more cyclic hydrocarbon, heteroaryl, or heterocyclic rings.

“Substituted aryl” is aryl having one or more non-interfering groups as substituents. For substitutions on a phenyl ring, the substituents may be in any orientation (i.e., ortho, meta or para).

“Heteroaryl” is an aryl group containing from one to four heteroatoms, preferably N, O, or S, or a combination thereof, which heteroaryl group is optionally substituted at carbon or nitrogen atom(s) with C1-6 alkyl, —CF₃, phenyl, benzyl, or thienyl, or a carbon atom in the heteroaryl group together with an oxygen atom form a carbonyl group, or which heteroaryl group is optionally fused with a phenyl ring. Heteroaryl rings may also be fused with one or, more cyclic hydrocarbon, heterocyclic, aryl, or heteroaryl rings. Heteroaryl includes, but is not limited to, 5-membered heteroaryls having one hetero atom (e.g., thiophenes, pyrroles, furans); 5-membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having 3 heteroatoms; 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heteroaryls with three heteroatoms (e.g., 1,3,5-triazine); and 6-membered heteroaryls with four heteroatoms.

“Substituted heteroaryl” is heteroaryl having one or more non-interfering groups as substituents.

“Heterocycle” or “heterocyclic” means one or more rings of 5-12 atoms, preferably 5-7 atoms, with or without unsaturation or aromatic character and at least one ring atom which is not carbon. Preferred heteroatoms include sulfur, oxygen, and nitrogen. Multiple rings may be fused, as in quinoline or benzofuran.

“Substituted heterocycle” is heterocycle having one or more side chains formed from non-interfering substituents.

“Non-interfering substituents are those groups that, when present in a molecule, are typically non-reactive with other functional groups contained within the molecule.

Suitable non-interfering substituents or radicals include, but are not limited to, halo, C1-C10 alkyl, C2-C10 alkenyl, C2-C10 alkynyl, C1-C10 alkoxy, C7-C12 aralkyl, C7-C12 alkaryl, C3-C10 cycloalkyl, C3-C10 cycloalkenyl, phenyl, substituted phenyl, toluoyl, xylenyl, biphenyl, C2-C12 alkoxyalkyl, C7-C12 alkoxyaryl, C7-C12 aryloxyalkyl, C6-C12 oxyaryl, C1-C6 alkylsulfinyl, C1-C10 alkylsulfonyl, —(CH₂)ₘ—O—(C1-C10 alkyl) wherein m is from 1 to 8, aryl, substituted aryl, substituted alkoxy, fluoroalkyl, heterocyclic radical, substituted heterocyclic radical, nitroalkyl, —NO₂, —CN, —NRC(O)—(C1-C10 alkyl), —C(O)—(C1-C10 alkyl), C2-C10 thioalkyl, —C(O)O—(C1-C10 alkyl), —OH, —SO₂, ═S, —COOH, —NR, carbonyl, —C(O)—(C1-C10 alkyl)-CF₃, —C(O)—CF₃, —C(O)NR₂, —(C1-C10 alkyl)-S—(C6-C12 aryl), —C(O)—(C6-C12 aryl), —(CH₂)ₘ—O—(CH₂)ₘ—O—(C1-C10 alkyl) wherein each m is from 1 to 8, —C(O)NR, —C(S)NR, —SO₂NR, —NRC(O)NR, —NRC(S)NR, salts thereof, and the like. Each R as used herein is H, alkyl or substituted alkyl, aryl or substituted aryl, aralkyl, or alkaryl.

“Heteroatom” means any non-carbon atom in a hydrocarbon analog compound. Examples include oxygen, sulfur, nitrogen, phosphorus, arsenic, silicon, selenium, tellurium, tin, and boron.

The term “drug”, “biologically active molecule”, “biologically active moiety” or “biologically active agent”, when used herein means any substance which can affect any physical or biochemical properties of a biological organism, including but not limited to viruses, bacteria, fungi, plants, animals, and humans. In particular, as used herein, biologically active molecules include any substance intended for diagnosis, cure mitigation, treatment, or prevention of disease in humans or other animals, or to otherwise enhance physical or mental well-being of humans or animals. Examples of biologically active molecules include, but are not limited to, peptides, proteins, enzymes, small molecule drugs, dyes, lipids, nucleosides, oligonucleotides, polynucleotides, nucleic acids, cells, viruses, liposomes, microparticles and micelles. Classes of biologically active agents that are suitable for use with the invention include, but are not limited to, antibiotics, fungicides, anti-viral agents, anti-inflammatory agents, anti-tumor agents, cardiovascular agents, anti-anxiety agents, hormones, growth factors, steroidal agents, and the like.

“Polyolefinic alcohol” refers to a polymer comprising a polyolefin backbone, such as polyethylene, having multiple pendant hydroxyl groups attached to the polymer backbone. An exemplary polyolefinic alcohol is polyvinyl alcohol.

As used herein, “non-peptidic” refers to a polymer backbone substantially free of peptide linkages. However, the polymer backbone may include a minor number of peptide linkages spaced along the length of the backbone, such as, for example, no more than about 1 peptide linkage per about 50 monomer units.

“Polypeptide” refers to any molecule comprising a series of amino acid residues, typically at least about 10-20 residues, linked through amide linkages (also referred to as peptide linkages) along the alpha carbon backbone. While in some cases the terms may be used synonymously herein, a polypeptide is a peptide typically having a molecular weight up to about 10,000 Da, while peptides having a molecular weight above that are commonly referred to as proteins. Modifications of the peptide side chains may be present, along with glycosylations, hydroxylations, and the like. Additionally, other non-peptidic molecules, including lipids and small drug molecules, may be attached to the polypeptide.

“Amino acid” refers to organic acids containing both a basic amine group and an acidic carboxyl group. The term encompasses essential and non-essential amino acids and both naturally occurring and synthetic or modified amino acids. The most common amino acids are listed herein by either their full name or by the three letter or single letter abbreviations: Glycine (Gly, G), Alanine (Ala, A), Valine (Val, V), Leucine (Leu, L), Isoleucine (Ile, I), Methionine (Met, M), Proline (Pro, P), Phenylalanine (Phe, F), Tryptophan (Trp, W), Serine (Ser, S), Threonine (Thr, T), Asparagine (Asn, N), Glutamine (Gln, Q), Tyrosine, (Tyr, Y), Cysteine (Cys, C), Lysine (Lys, K), Arginine (Arg, R), Histidine (His, H), Aspartic Acid (Asp, D), and Glutamic acid (Glu, E).

By “residue” is meant the portion of a molecule remaining after reaction with one or more molecules. For example, a PKC inhibitor residue in the polymer conjugate of the invention is the portion of a PKC inhibitor remaining following covalent linkage to a polymer backbone.

“Oligomer” refers to short monomer chains comprising 2 to about 10 monomer units, preferably 2 to about 5 monomer units.

The term “conjugate” is intended to refer to the entity formed as a result of covalent attachment of a molecule, e.g., a biologically active molecule such as a PKC inhibitor, to a reactive polymer molecule, preferably poly(ethylene glycol).

“Bifunctional” in the context of a polymer of the invention refers to a polymer possessing two reactive functional groups which may be the same or different.

“Multifunctional” in the context of a polymer of the invention means a polymer having 3 or more functional groups attached thereto, where the functional groups may be the same or different. Multifunctional polymers of the invention will typically comprise from about 3-100 functional groups, or from 3-50 functional groups, or from 3-25 functional groups, or from 3-15 functional groups, or from 3 to 10 functional groups, or will contain 3, 4, 5, 6, 7, 8, 9 or 10 functional groups attached to the polymer backbone.

II. The Polymer Conjugate

As described generally above, the polymer conjugates of the invention comprise a water-soluble and non-peptidic polymer covalently attached to a PKC inhibitor, such as a bisindolylmaleimide. Where the PKC inhibitor is a bisindolylmaleimide, the polymer can be attached to any carbon atom of either indole ring or the nitrogen atom of the maleimide group. The conjugates of the invention can comprise a single polymer attached to the PKC inhibitor molecule or multiple polymers attached to the PKC inhibitor. The polymer conjugates of the invention are useful for the treatment or prophylaxis of any PKC mediated disease or disorder, such as various inflammatory diseases and conditions, immunological diseases, bronchopulmonary diseases (e.g., asthma), cardiovascular diseases, diabetes, dermatological diseases (e.g., psoriasis), cancer, and central nervous system (CNS) diseases (e.g., Alzheimer's disease).

Typically, the number average molecular weight of the polymer portion of a polymer conjugate of the invention is about 100 Da to about 100,000 Da, preferably about 1,000 Da to about 50,000 Da, more preferably about 5,000 Da to about 30,000 Da. Polymer backbones having a number average molecular weight of about 500 Da, about 800 Da, about 900 Da, about 1,000 Da, about 2,000 Da, about 3,000 Da, about 4,000 Da, about 5,000 Da, about 10,000 Da, about 15,000 Da, about 20,000 and about 25,000 Da are particularly preferred.

The conjugates of the invention are preferably prodrugs, meaning the linkage between the polymer backbone and the PKC inhibitor is hydrolytically degradable so that the PKC inhibitor parent molecule is released into circulation following administration to a patient. Exemplary degradable linkages include carboxylate ester, phosphate ester, anhydrides, acetals, ketals, acyloxyalkyl ether, imines, orthoesters, peptides, and oligonucleotides. However, a hydrolytically stable linkage, such as amide, urethane (also known as carbamate), amine, thioether (also known as sulfide), and urea (also known as carbamide) linkages, can also be used without departing from the invention. The particular linkage and linkage chemistry employed will depend upon the subject PKC inhibitor molecule, functional groups within the molecule available either for attachment to a polymer or conversion to a suitable attachment site, the presence of additional functional groups within the molecule, and the like, and can be readily determined by one skilled in the art based upon the guidance presented herein.

The polymer conjugates of the invention maintain at least a measurable degree of PKC inhibition activity. That is to say, a polymer conjugate in accordance with the invention will possesses anywhere from about 1% to about 100% or more of the specific activity of the unmodified parent PKC inhibitor compound. Such activity may be determined using a suitable in-vivo or in-vitro model, depending upon the known activity of the particular PKC inhibitor parent compound. For example, in-vitro assays using purified rat brain PKC or human neutrophil PKC can be used as described in Davis et al., FEBS Lett. 259(1):61-63 (1989). In general, a polymer conjugate of the invention will possess a specific activity of at least about 2%, 5%, 10%, 15%, 25%, 30%, 40%, 50%, 60%, 80%, 90% or more relative to that of the unmodified parent PKC inhibitor, when measured in a suitable model, such as those well known in the art. Preferably, a conjugate of the invention will maintain at least 50% or more of the PKC inhibition activity of the unmodified parent compound.

A polymer conjugate of the invention will typically comprise a water-soluble and non-peptidic polymer, such as poly(ethylene glycol), covalently attached to a bisindolylmaleimide or other PKC inhibitor compound, and have a generalized structure as shown below:

POLY-X—I_{PKC} Formula I

wherein:

POLY is a water-soluble and non-peptidic polymer;

X is a linkage, preferably a hydrolytically degradable linkage, covalently attaching the polymer to the PKC inhibitor molecule; and

I_{PKC} is the PKC inhibitor molecule, such as a bisindolylmaleimide.

The polymer conjugates of the invention may be administered per se or in the form of a pharmaceutically acceptable salt, and any reference to the polymer conjugates of the invention herein is intended to include pharmaceutically acceptable salts. If used, a salt of the polymer conjugate should be both pharmacologically and pharmaceutically acceptable, but non-pharmaceutically acceptable salts may conveniently be used to prepare the free active compound or pharmaceutically acceptable salts thereof and are not excluded from the scope of this invention. Such pharmacologically and pharmaceutically acceptable salts can be prepared by reaction of the polymer conjugate with an organic or inorganic acid, using standard methods detailed in the literature. Examples of useful salts include, but are not limited to, those prepared from the following acids: hydrochloric, hydrobromic, sulfuric, nitric, phosphoric, maleic, acetic, salicyclic, p-toluenesulfonic, tartaric, citric, methanesulphonic, formic, malonic, succinic, naphthalene-2-sulphonic and benzenesulphonic, and the like. Also, pharmaceutically acceptable salts can be prepared as alkaline metal or alkaline earth salts, such as sodium, potassium, or calcium salts of a carboxylic acid group.

A. Polymer Backbone

In general, the water soluble and non-peptidic polymer portion of the conjugate should be non-toxic and biocompatible, meaning that the polymer is capable of coexistence with living tissues or organisms without causing harm. When referring to a polymer conjugate, it is to be understood that the polymer can be any of a number of water soluble and non-peptidic polymers, such as those described herein as suitable for use in the present invention. Preferably, poly(ethylene glycol) (PEG) is the polymer backbone. The term PEG includes poly(ethylene glycol) in any of a number of geometries or forms, including linear forms (e.g., alkoxy PEG or bifunctional PEG), branched or multi-arm forms (e.g., forked PEG or PEG attached to a polyol core), pendant PEG, or PEG with degradable linkages therein, to be more fully described below.

In its simplest form, PEG has the formula

—CH₂CH₂O—(CH₂CH₂O)ₙ—CH₂CH₂— Formula II

wherein n is from about 2 to about 2,000, typically from about 20 to about 1,000.

End-capped polymers, meaning polymers having at least one terminus capped with a relatively inert group (e.g., an alkoxy group), can be used as a polymer of the invention. For example, methoxy-PEG-OH, or mPEG in brief, is a form of PEG wherein one terminus of the polymer is a methoxy group, while the other terminus is a hydroxyl group that is subject to ready chemical modification. The structure of mPEG is given below.

CH₃O—(CH₂CH₂O)ₙ—CH₂CH₂—OH Formula III

wherein n is as described above.

Multi-armed or branched PEG molecules, such as those described in U.S. Pat. No. 5,932,462, which is incorporated by reference herein in its entirety, can also be used as the PEG polymer. Generally speaking, a multi-armed or branched polymer possesses two or more polymer “arms” extending from a central branch point (e.g., C in the structure below) that is covalently attached, either directly or indirectly via intervening connecting atoms, to one active moiety, such as a PKC inhibitor. For example, an exemplary branched PEG polymer can have the structure:

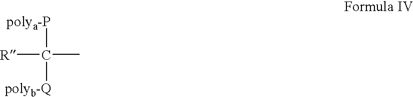

wherein:

polyₐ and poly_{b} are PEG backbones, such as methoxy poly(ethylene glycol);

R″ is a nonreactive moiety, such as H, methyl or a PEG backbone; and

P and Q are nonreactive linkages. In a preferred embodiment, the branched PEG polymer is methoxy poly(ethylene glycol) disubstituted lysine.

The PEG polymer may alternatively comprise a forked PEG. Generally speaking, a polymer having a forked structure is characterized as having a polymer chain attached to two or more active agents via covalent linkages extending from a hydrolytically stable branch point in the polymer. An example of a forked PEG is represented by PEG-YCHZ₂, where Y is a linking group and Z is an activated terminal group for covalent attachment to a biologically active agent, such as a PKC inhibitor. The Z group is linked to CH by a chain of atoms of defined length. International Application No. PCT/US99/05333, the contents of which are incorporated by reference herein, discloses various forked PEG structures capable of use in the present invention. The chain of atoms linking the Z functional groups to the branching carbon atom serve as a tethering group and may comprise, for example, an alkyl chain, ether linkage, ester linkage, amide linkage, or combinations thereof.

The PEG polymer may comprise a pendant PEG molecule having reactive groups, such as carboxyl, covalently attached along the length of the PEG backbone rather than at the end of the PEG chain. The pendant reactive groups can be attached to the PEG backbone directly or through a linking moiety, such as an alkylene group.

In addition to the above-described forms of PEG, the polymer can also be prepared with one,,or more weak or degradable linkages in the polymer backbone, including any of the above described polymers. For example, PEG can be prepared with ester linkages in the polymer backbone that are subject to hydrolysis. As shown below, this hydrolysis results in cleavage of the polymer into fragments of lower molecular weight:

-PEG-CO₂-PEG-+H₂O→-PEG-CO₂H+HO-PEG-

Other hydrolytically degradable linkages, useful as a degradable linkage within a polymer backbone, include carbonate linkages; imine linkages resulting, for example, from reaction of an amine and an aldehyde (see, e.g., Ouchi et al., Polymer Preprints, 38(1):582-3 (1997), which is incorporated herein by reference.); phosphate ester linkages formed, for example, by reacting an alcohol with a phosphate group; hydrazone linkages which are typically formed by reaction of a hydrazide and an aldehyde; acetal linkages that are typically formed by reaction between an aldehyde and an alcohol; ortho ester linkages that are, for example, formed by reaction between a formate and an alcohol; peptide linkages formed by an amine group, e.g., at an end of a polymer such as PEG, and a carboxyl group of a peptide; and oligonucleotide linkages formed by, for example, a phosphoramidite group, e.g., at the end of a polymer, and a 5′ hydroxyl group of an oligonucleotide.

It is understood by those skilled in the art that the term poly(ethylene glycol) or PEG represents or includes all the above forms of PEG.

Any of a variety of monofunctional, bifunctional or multifunctional polymers that are non-peptidic and water-soluble can also be used to form a conjugate in accordance with the present invention. The polymer backbone can be linear, or can be in any of the above-described forms (e.g., branched, forked, and the like). Examples of suitable polymers include, but are not limited to, other poly(alkylene glycols), copolymers of ethylene glycol and propylene glycol, poly(olefinic alcohol), poly(vinylpyrrolidone), poly(hydroxyalkylmethacrylamide), poly(hydroxyalkylmethacrylate), poly(saccharides), poly(α-hydroxy acid), poly(acrylic acid), poly(vinyl alcohol), polyphosphazene, polyoxazoline, poly(N-acryloylmorpholine), such as described in U.S. Pat. No. 5,629,384, which is incorporated by reference herein in its entirety, and copolymers, terpolymers, and mixtures thereof.

B. Linkage Between Polymer and PKC Inhibitor

The linkage between the PKC inhibitor and the polymer backbone (i.e., X in Formula I) results from the reaction of a reactive functional group of the polymer with a functional group on the PKC inhibitor molecule, such as a bisindolylmaleimide molecule. The specific linkage will depend on the structure of the functional groups utilized, and will typically be governed by the functional groups contained in the PKC inhibitor molecule. For example, an amide linkage can be formed by reaction of a polymer having a terminal carboxylic acid group, or an active ester thereof, in the presence of a coupling agent, such as DCC, DMAP, or HOBT, with a PKC inhibitor having an amine group. Alternatively, a sulfide linkage can be formed by reaction of a polymer terminated with a thiol group with a PKC inhibitor bearing a hydroxyl group. In another embodiment, an amine linkage is formed by reaction of an amino-terminated polymer with a PKC inhibitor bearing a hydroxyl group. In yet another embodiment, a polymer having a terminal carboxylic acid is reacted with a PKC inhibitor bearing a hydroxyl group in the presence of a coupling agent to form an ester linkage. The particular coupling chemistry employed will depend upon the structure of the PKC inhibitor, the potential presence of multiple functional groups within the PKC inhibitor, the need for protection/deprotection steps, chemical stability of the molecule, and the like, and will be readily determined by one skilled in the art. Illustrative linking chemistry useful for preparing the polymer conjugates of the invention can be found, for example, in Wong, S. H., (1991), “Chemistry of Protein Conjugation and Crosslinking”, CRC Press, Boca Raton, Fla. and in Brinkley, M. (1992) “A Brief Survey of Methods for Preparing Protein Conjugates with Dyes, Haptens, and Crosslinking Reagents”, in Bioconjug. Chem., 3, 2013.

The linkage is preferably hydrolytically degradable so that the PKC inhibitor is released into circulation over time after administration to the patient. Exemplary hydrolytically degradable linkages include carboxylate ester, phosphate ester, anhydrides, acetals, ketals, acyloxyalkyl ether, imines, orthoesters, peptides and oligonucleotides. If desired, a hydrolytically stable linkage, such as amide, urethane (also known as carbamate), amine, thioether (also known as sulfide), and urea (also known as carbamide) linkages, can also be used without departing from the invention. The overall X linkage is intended to encompass any linkage between the polymer and the PKC inhibitor molecule having an overall length of from 1 to about 20 atoms, preferably 1 to about 10 atoms. In one embodiment, the X linkage is —CONH—, —C(O)—, —O—(CH₂)ₙ—C(O)—O— where n is 1-10, —O—(CH₂)ₙ—C(O)—NH— wherein n is 1-10, —C(O)—O—(CH₂)ₙ—C(O)—NH— where n is 1-10, or —O—CH₂—C(O)O—CH₂—C(O)—NH—.

C. PKC Inhibitor

As used herein, the term “PKC inhibitor” refers to any molecule that inhibits the function of any isozyme of protein kinase C, particularly those that selectively inhibit specific PKC isozymes, such as the alpha, beta, or gamma isozymes. The PKC inhibitor molecule can be any PKC inhibitor known in the art, including any of a variety of bisindolylmaleimide compounds or indazolyl-substituted pyrroline compounds, such as those compounds disclosed in the following references, all of which are incorporated by reference herein in their entirety: Davis et al., FEBS Lett. 259(1):61-63 (1989); Twomey et al., Biochem. Biophys. Res. Commun. 171(3): 1087-1092 (1990); Toullec, et al., J. Biol. Chem. 266(24): 15771-15781 (1991); Davis et al., J. Med. Chem. 35:994-1001 (1992); Bit et al., J. Med. Chem. 36:21-29 (1993); U.S. Pat. Nos. 5,057,614, 5,936,084, and 6,284,783; International Publication Nos. WO 98/04551, WO 99/44606, WO 99/44607, WO 99/47518, and WO 02/46183; EP 0 940 141 A2.

In one embodiment, the PKC inhibitor is a bisindolylmaleimide having the structure:

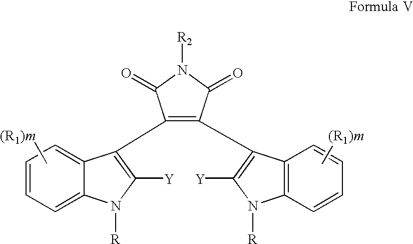

wherein:

each R is independently selected from the group consisting of alkyl, substituted alkyl, cycloalkyl, substituted cycloalkyl, alkenyl, alkynyl, aryl, substituted aryl, heteroaryl, substituted heteroaryl, heterocycle, and substituted heterocycle, or both R groups together form -T-W-J-, wherein W is —O—, —S—, —SO—, —SO₂—, —CO—, C2-C6alkylene, substituted C2-C6alkylene, C2-C6alkenylene, -arylene-, -arylene-alkylene-O—, -heterocycle-, -heterocycle-alkylene-O—, -cycloalkyl-alkylene-O—, —NR₃—, —NOR₃—, —CONH—, or —NHCO— (where R₃ is hydrogen, alkyl, substituted alkyl, —C(O)O-alkyl, aminocarbonyl, amidino, alkylsulphinyl, aminosulphonyl, or alkylsulphonyl), and T and J are independently C1-C6alkylene or substituted. C1-C6alkylene, or T, W, and J together form —C2-C6alkylene-AA-, where AA is an amino acid residue;

each R₁ is independently selected from the group consisting of halo, hydroxy, alkyl, substituted alkyl (e.g., alkyl substituted with one or more halo), alkoxy, substituted alkoxy, aryloxy, substituted aryloxy, nitro, thiol, amino, substituted amino (e.g., acylamino, monoalkylamino, dialkylamino, —NHC(O)alkyl), alkylsulphinyl, alkylsulphonyl, and alkylthio;

m is 0-4 (e.g., 0, 1, 2, 3, or 4);

R₂ is selected from the group consisting of hydrogen, halo, hydroxy, alkyl, substituted alkyl, alkoxy, substituted alkoxy, amino, substituted amino (e.g., —NHC(O)alkyl, alkylamino, dialkyamino), and alkylcarbonyl (i.e., —C(O)alkyl); and

each Y is independently selected from the group consisting of hydrogen, alkyl, substituted alkyl (e.g., aralkyl, alkoxyalkyl, hydroxyalkyl, haloalkyl, aminoalkyl, monoalkylaminoalkyl, dialkylaminoalkyl, acylaminoalkyl, alkylsulphonylaminoalkyl, arylsulphonylaminoalkyl, mercaptoalkyl, alkylthioalkyl, carboxyalkyl, alkoxycarbonylalkyl, and aminocarbonylalkyl), alkylthio, and alkylsulphinyl, or Y together with R, form a fused C3-C8 heterocyclic ring, optionally substituted with one or more alkyl, substituted alkyl (e.g., aminoalkyl, alkylaminoalkyl, dialkylaminoalkyl), or amino groups.

In one embodiment, Y and R₂ are hydrogen and each R is independently hydrogen, alkyl, haloalkyl, hydroxyalkyl, alkoxyalkyl, alkylamino, alkylaminoalkyl, dialkylaminoalkyl, trialkylaminoalkyl, aminoalkylaminoalkyl, azidoalkyl, acylaminoalkyl, acylthioalkyl, alkylsulphonylaminoalkyl, arylsulphonylaminoalkyl, mercaptoalkyl, alkylthioalkyl, alkylsuphinylalkyl, alkylsulphonylalkyl, alkylsulphonyloxyalkyl, alkylcarbonyloxyalkyl, cyanoalkyl, amidinoalkyl, isothiocyanatoalkyl, glucopyranosyl, carboxyalkyl, alkoxycarbonylalkyl, aminocarbonylalkyl, hydroxyalkylthioalkyl, mercaptoalkylthioalkyl, arylthioalkyl, carboxyalkylthioalkyl, alkyl-S(C═NH)NH₂, or alkyl-NC(═NNO₂)NH₂.

In a particularly preferred embodiment, the PKC inhibitor molecule has the structure:

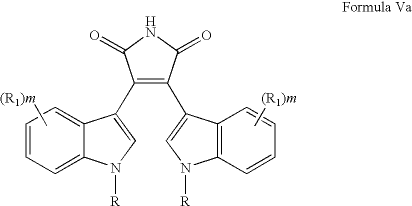

wherein:

each R is independently hydrogen, alkyl, haloalkyl, hydroxyalkyl, alkoxyalkyl, alkylamino, alkylaminoalkyl, dialkylaminoalkyl, trialkylaminoalkyl, aminoalkylaminoalkyl, azidoalkyl, acylaminoalkyl, acylthioalkyl, alkylsulphonylaminoalkyl, arylsulphonylaminoalkyl, mercaptoalkyl, alkylthioalkyl, alkylsuphinylalkyl, alkylsulphonylalkyl, alkylsulphonyloxyalkyl, alkylcarbonyloxyalkyl, cyanoalkyl, amidinoalkyl, isothiocyanatoalkyl, glucopyranosyl, carboxyalkyl, alkoxycarbonylalkyl, aminocarbonylalkyl, hydroxyalkylthioalkyl, mercaptoalkylthioalkyl, arylthioalkyl, carboxyalkylthioalkyl, alkyl-S(C═NH)NH₂, or alkyl-NC(═NNO₂)NH₂;

each R₁ is independently selected from the group consisting of halo, hydroxy, alkyl, haloalkyl, alkoxy, aryloxy, nitro, thiol, amino, acylamino, monoalkylamino, dialkylamino, —NHC(O)alkyl, alkylsulphinyl, alkylsulphonyl, and alkylthio; and

m is 0-4 (e.g., 0, 1, 2, 3, or 4).

Exemplary PKC inhibitor compounds include:

3,4-bis(indol-3-yl)-1H-pyrrole-2,5-dione,

3,4-bis(1-methyl-indol-3-yl)-1H-pyrrole-2,5-dione,

3-[1-(3-hydroxypropyl)-indol-3-yl]-4-(1-methyl-indol-3-yl)-1H-pyrrole-2,5-dione,

3-[1-(3-aminopropyl)-indol-3-yl]-4-(1-methyl-indol-3-yl)-1H-pyrrole-2,5-dione,

3-[1-[3-(methylamino)propyl]-indol-3-yl]-4-(1-methyl-indol-3-yl)-1H-pyrrole-2,5-dione,

3-[1-[3-(dimethylamino)propyl]-indol-3-yl]-4-(1-methyl-indol-3-yl)-1H-pyrrole-2,5-dione,

3-[1-[3-(amidinothio)propyl]-indol-3-yl]-4-(1-methyl-indol-3-yl)-1H-pyrrole-2,5-dione,

3-(1-methyl-indol-3-yl)-4-[1-[3-(2-nitroguanidino)propyl]-indol-3-yl]-1H-pyrrole-2,5-dione,

3-[1-(3-guanidinopropyl)-indol-3-yl]-4-(1-methyl-indol-3-yl)-1H-pyrrole-2,5-dione,

3-[l -(3-isothiocyanatopropyl)-indol-3-yl]-4-(1-methyl-indol-3-yl)-1H-pyrrole-2,5-dione,

3-[1-(4-amidinobutyl)-indol-3-yl]-4-(1-methyl-indol-3-yl)-1H-pyrrole-2,5-dione,

3-[6,7,8,9-tetrahydropyrido[1,2-a]indol-10-yl]-4-(1-methyl-indol-3-yl)-1H-pyrrole-2,5-dione,

3-[8-(aminomethyl)-6,7,8,9-tetrahydropyrido[1,2-a]indol-10-yl]-4-(1-methyl-indol-3-yl-)-1H-pyrrole-2,5-dione,

3-[1-[3-(dimethylamino)propyl]-indol-3-yl]-4-(indol-3-yl)-1H-pyrrole-2,5-dione,

3-(1-methyl-6-nitro-indol-3-yl)-4-(indol-3-yl)-1H-pyrrole-2,5-dione,

3-(1-methyl-6-nitro-indol-3-yl)-4-(1-hydroxymethyl-indol-3-yl)-1H-pyrrole-2,5-dione,

3-(1-methyl-indol-3-yl)-4-(6-nitro-indol-3-yl)-1H-pyrrole-2,5-dione,

3-(1-methyl-6-methoxy-indol-3-yl)-4-(1-methyl-6-nitro-indol-3-yl)-1H-pyrrole-2,5-dione,

3-(1-methyl-6-methylsulphanyl-indol-3-yl)-4-(1-methyl-6-nitro-indol-3-yl)-1H-pyrrole-2,5-dione,

3-(1-methyl-6-hydroxy-indol-3-yl)-4-(1-methyl-indol-3-yl)-1H-pyrrole-2,5-dione,

3-(1-methyl-6-amino-indol-3-yl)-4-(1-methyl-indol-3-yl)-1H-pyrrole-2,5-dione,

3-(1-methyl-6-amino-indol-3-yl)-4-(1-methyl-6-nitro-indol-3-yl)-1H-pyrrole-2,5-dione,

3-(1-methyl-indol-3-yl)-4-(1-methyl-6-nitro-indol-3-yl)-1H-pyrrole-2,5-dione, and

(S)-3,4-[N,N′-1,1′-((2″-ethoxy)-3′″(O)-4′″-(N,N-dimethylamino)-butane)-bis-(3,3′-indolyl)]-1H-pyrrole-2,5-dione.

The PKC molecule can be synthesized using methodology disclosed in the references cited above. For example, bisindolylmaleimide molecules useful in the present invention can be synthesized as described by Brenner, et al., in Tetrahedron 44:2887-2892 (1988). As described therein, a Grignard reaction between a dibromo-substituted maleimide and indolyl-MgBr results in formation of a bisindolylmaleimide. The maleimide and indole starting reagents are either commercially available or can be prepared using methods known in the art.

D. Method of Forming Polymer Conjugates of PKC Inhibitors

The polymer conjugate of the invention can be formed using known techniques for covalent attachment of an activated polymer, such as an activated PEG, to a biologically active agent (See, for example, POLY(ETHYLENE GLYCOL) CHEMISTRY AND BIOLOGICAL APPLICATIONS, American Chemical Society, Washington, D.C. (1997)). The general method involves selection of a reactive polymer bearing a functional group suitable for reaction with a functional group of the PKC inhibitor, such as a bisindolylmaleimide molecule, and reaction of the reactive polymer with the PKC inhibitor in solution to form a covalently bonded conjugate.

Selection of the functional group of the polymer will depend, in part, on the functional group on the PKC inhibitor molecule. The functional group of the polymer is preferably chosen to result in formation of a hydrolytically degradable linkage between the PKC inhibitor and the polymer. A polymer of the invention suitable for coupling to a PKC inhibitor molecule will typically have a terminal functional group such as the following: N-succinimidyl carbonate (see e.g., U.S. Pat. Nos. 5,281,698, 5,468,478), amine (see, e.g., Buckmann et al. Makromol. Chem. 182:1379 (1981), Zalipsky et al. Eur. Polym. J. 19:1177 (1983)), hydrazide (See, e.g., Andresz et al. Makromol. Chem. 179:301 (1978)), succinimidyl propionate and succinimidyl butanoate (see, e.g., Olson et al. in Poly(ethylene glycol) Chemistry & Biological Applications, pp 170-181, Harris & Zalipsky Eds., ACS, Washington, D.C., 1997; see also U.S. Pat. No. 5,672,662), succinimidyl succinate (See, e.g., Abuchowski et al. Cancer Biochem. Biophys. 7:175 (1984) and Joppich et al., Makromol. Chem. 180:1381 (1979), succinimidyl ester (see, e.g., U.S. Pat. No. 4,670,417), benzotriazole carbonate (see, e.g., U.S. Pat. No. 5,650,234), glycidyl ether (see, e.g., Pitha et al. Eur. J. Biochem. 94:11 (1979), Elling et al., Biotech. Appl. Biochem. 13:354 (1991), oxycarbonylimidazole (see, e.g., Beauchamp, et al., Anal. Biochem. 131:25 (1983), Tondelli et al. J. Controlled Release 1:251 (1985)), p-nitrophenyl carbonate (see, e.g., Veronese, et al., Appl. Biochem. Biotech., 11:141 (1985); and Sartore et al., Appl. Biochem. Biotech., 27:45 (1991)), aldehyde (see, e.g., Harris et al. J. Polym. Sci. Chem. Ed. 22:341 (1984), U.S. Pat. No. 5,824,784, U.S. Pat. No. 5,252,714), maleimide (see, e.g., Goodson et al. Bio/Technology 8:343 (1990), Romani et al. in Chemistry of Peptides and Proteins 2:29 (1984)), and Kogan, Synthetic Comm. 22:2417 (1992)), orthopyridyl-disulfide (see, e.g., Woghiren, et al. Bioconj. Chem. 4:314 (1993)), acrylol (see, e.g., Sawhney et al., Macromolecules, 26:581 (1993)), vinylsulfone (see, e.g., U.S. Pat. No. 5,900,461). All of the above references are incorporated herein by reference.

In an embodiment exemplified in Examples 1-3, a carboxylic acid terminated polymer is reacted with a hydroxyl group on a PKC inhibitor molecule to form an ester linkage therebetween. In another embodiment illustrated in Examples 4-6, a carboxylic acid terminated polymer is reacted with an amino group on the PKC inhibitor molecule to form an amide linkage. In yet another embodiment exemplified in Examples 7-8, a polymer terminated with an acid halide is reacted with the nitrogen atom of the maleimide ring of a lithium salt of a bisindolylmaleimide compound to form an amide linkage.

The polymer conjugate product may be purified and collected using methods known in the art for biologically active conjugates of this type. Typically, the polymer conjugate is isolated by precipitation followed by filtration and drying.

E. Exemplary Conjugate Structures

More specific structural embodiments of the conjugates of the invention will now be described, all of which are intended to be encompassed by the structure of Formula I above. The specific structures shown below are presented as exemplary structures only, and are not intended to limit the scope of the invention.

An embodiment of a linear polymer of the invention can be structurally represented as shown below:

Z-POLY-X—I_{PKC} Formula Ia

Wherein Z is a capping group or a functional group, POLY is a water soluble and non-peptidic polymer backbone, and X and I_{PKC} are as defined above. In a preferred embodiment, Z is methoxy, POLY is poly(ethylene glycol), X is a hydrolytically degradable linkage, and I_{PKC} has the structure shown in Formula V or Formula Va above.

The Z group can be a relatively inert capping group, such as alkoxy (e.g. methoxy or ethoxy), alkyl, benzyl, aryl, or aryloxy (e.g. benzyloxy). Alternatively, the Z group can be a functional group capable of readily reacting with a functional group on a biologically active molecule, such as another bisindolylmaleimide or other PKC inhibitor.

Exemplary functional groups include hydroxyl, active ester (e.g., N-hydroxysuccinimidyl ester or 1-benzotriazolyl ester), active carbonate (e.g., N-hydroxysuccinimidyl carbonate and 1-benzotriazolyl carbonate), acetal, aldehyde, aldehyde hydrate, alkenyl, acrylate, methacrylate, acrylamide, active sulfone, amine, hydrazide, thiol, carboxylic acid, isocyanate, isothiocyanate, maleimide, vinylsulfone, dithiopyridine, vinylpyridine, iodoacetamide, epoxide, glyoxal, dione, mesylate, tosylate, or tresylate.

In a homobifunctional embodiment of Formula Ia, Z has the structure —X—I_{PKC}, wherein X and I_{PKC} are as defined above.

One example of a multi-arm embodiment of the polymer conjugate of the invention has the structure:

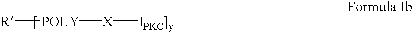

wherein

each POLY is a water soluble and non-peptidic polymer backbone, R′ is a central core molecule, y is from about 3 to about 100, preferably 3 to about 25, and X and I_{PKC} are as defined above. The core moiety, R′, is a residue of a molecule selected from the group consisting of polyols, polyamines, and molecules having a combination of alcohol and amine groups. Specific examples of central core molecules include glycerol, glycerol oligomers, pentaerythritol, sorbitol, and lysine.

The central core molecule is preferably a residue of a polyol having at least three hydroxyl groups available for polymer attachment. A “polyol” is a molecule comprising a plurality of available hydroxyl groups. Depending on the desired number of polymer arms, the polyol will typically comprise 3 to about 25 hydroxyl groups. The polyol may include other protected or unprotected functional groups as well without departing from the invention. Although the spacing between hydroxyl groups will vary from polyol to polyol, there are typically 1 to about 20 atoms, such as carbon atoms, between each hydroxyl group, preferably 1 to about 5. Preferred polyols include glycerol, reducing sugars such as sorbitol, pentaerythritol, and glycerol oligomers, such as hexaglycerol. A 21-arm polymer can be synthesized using hydroxypropyl-β-cyclodextrin, which has 21 available hydroxyl groups. The particular polyol chosen will depend on the desired number of hydroxyl groups needed for attachment to the polymer arms.

As noted above, the point of attachment between POLY-X— and the PKC inhibitor (I_{PKC}) in either Formula Ia or Ib can be any carbon atom of either indole ring or the nitrogen atom of the maleimide ring. For example, where I_{PKC} is a compound of Formula Va above, a polymer conjugate embodiment of Formula Ia comprising a single polymer can have either of the following structures:

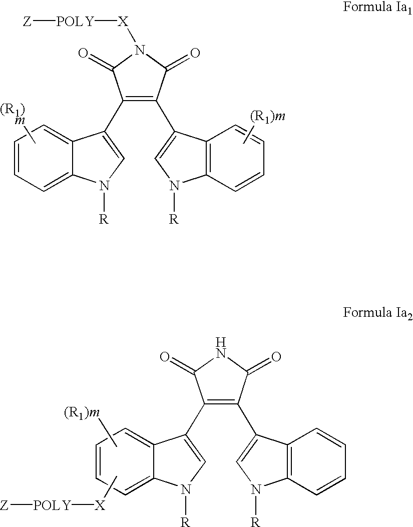

wherein

R, R₁, m, POLY, X, and Z are as defined above. As would be understood, the invention also includes analogous conjugate structures where POLY-X— is attached to any carbon atom of the indole rings or to the nitrogen atom of the maleimide ring of a bisindolylmaleimide of Formula V. Similarly, the invention includes conjugate structures of Formula Ib where POLY-X— is attached to any carbon atom of the indole rings or the nitrogen atom of the maleimide ring of a bisindolylmaleimide, for example a bisindolylmaleimide of Formula V or Va above. More than one polymer could be attached to the PKC inhibitor. For example, two polymers could be attached to the indole rings of a bisindolylmaleimide or one polymer could be attached to the nitrogen atom of the maleimide ring and one polymer could be attached to an indole ring.

III. Pharmaceutical Compositions Including a Polymer Conjugate of the Invention

The invention provides pharmaceutical formulations or compositions, both for veterinary and for human medical use, which comprise one or more polymer conjugates of the invention or a pharmaceutically acceptable salt thereof, with one or more pharmaceutically acceptable carriers, and optionally any other therapeutic ingredients, stabilizers, or the like. The carrier(s) must be pharmaceutically acceptable in the sense of being compatible with the other ingredients of the formulation and not unduly deleterious to the recipient thereof. The compositions of the invention may also include polymeric excipients/additives or carriers, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin. The compositions may further include diluents, buffers, binders, disintegrants, thickeners, lubricants, preservatives (including antioxidants), flavoring agents, taste-masking agents, inorganic salts (e.g., sodium chloride), antimicrobial agents (e.g., benzalkonium chloride), sweeteners, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations). Other pharmaceutical excipients and/or additives suitable for use in the compositions according to the invention are listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), and in “Handbook of Pharmaceutical Excipients”, Third Ed., Ed. A. H. Kibbe, Pharmaceutical Press, 2000.

The conjugates of the invention may be formulated in compositions including those suitable for oral, rectal, topical, nasal, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing the active agent or compound (i.e., the polymer conjugate) into association with a carrier that constitutes one or more accessory ingredients. In general, the compositions are prepared by bringing the active compound into association with a liquid carrier to form a solution or a suspension, or alternatively, bring the active compound into association with formulation components suitable for forming a solid, optionally a particulate product, and then, if warranted, shaping the product into a desired delivery form. Solid formulations of the invention, when particulate, will typically comprise particles with sizes ranging from about 1 nanometer to about 500 microns. In general, for solid formulations intended for intravenous administration, particles will typically range from about 1 nm to about 10 microns in diameter.

The amount of polymer conjugate in the formulation will vary depending upon the specific PKC inhibitor employed, its activity in conjugated form, the molecular weight of the conjugate, and other factors such as dosage form, target patient population, and other considerations, and will generally be readily determined by one skilled in the art. The amount of conjugate in the formulation will be that amount necessary to deliver a therapeutically effective amount of PKC inhibitor to a patient in need thereof to achieve at least one of the therapeutic effects associated with the PKC inhibitor. In practice, this will vary widely depending upon the particular conjugate, its activity, the severity of the condition to be treated, the patient population, the stability of the formulation, and the like. Compositions will generally contain anywhere from about 1% by weight to about 99% by weight conjugate, typically from about 2% to about 95% by weight conjugate, and more typically from about 5% to 85% by weight conjugate, and will also depend upon the relative amounts of excipients/additives contained in the composition. More specifically, the composition will typically contain at least about one of the following percentages of conjugate: 2%, 5%, 10%, 20%, 30%, 40%, 50%, 60%, or more by weight.

Compositions of the present invention suitable for oral administration may be presented as discrete units such as capsules, cachets, tablets, lozenges, and the like, each containing a predetermined amount of the active agent as a powder or granules; or a suspension in an aqueous liquor or non-aqueous liquid such as a syrup, an elixir, an emulsion, a draught, and the like.

A tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tablets comprised with a suitable carrier may be made by molding in a suitable machine.

A syrup may be made by adding the active compound to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

Formulations suitable for parenteral administration conveniently comprise a sterile aqueous preparation of the conjugate, which can be formulated to be isotonic with the blood of the recipient.

Nasal spray formulations comprise purified aqueous solutions of the active agent with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media such as mineral oil, petroleum, polyhydroxy alcohols or other bases used for topical formulations. The addition of other accessory ingredients as noted above may be desirable.

Pharmaceutical formulations are also provided which are suitable for administration as an aerosol, by inhalation. These formulations comprise a solution or suspension of the desired polymer conjugate or a salt thereof. The desired formulation may be placed in a small chamber and nebulized. Nebulization may be accomplished by compressed air or by ultrasonic energy to form a plurality of liquid droplets or solid particles comprising the conjugates or salts thereof.

IV. Method of Using the Polymer Conjugates of the Invention

The polymer conjugates of the invention can be used to treat any condition responsive to PKC inhibitors in any animal, particularly in mammals, including humans. See, generally, U.S. Pat. Nos. 5,936,084 and 5,057,614; WO 02/46183. Exemplary conditions include viral infections such as cytomegalovirus (CMV) infections (See EP 0 940 141 A2), inflammatory diseases and conditions, immunological diseases, bronchopulmonary diseases such as asthma (See WO 99/44606), cardiovascular diseases, diabetes, dermatological diseases (e.g., psoriasis), cancer, and central nervous system (CNS) diseases (e.g., Alzheimer's disease). The anti-tumor activity of the polymer conjugates of the invention is derived from the ability to induce apoptosis (See U.S. Pat. No. 6,284,783) and the ability to inhibit cell proliferation (See WO 98/04551 and WO 99/47518).

The method of treatment comprises administering to the mammal a therapeutically effective amount of a polymer conjugate of a PKC inhibitor as described above. The therapeutically effective dosage amount of any specific conjugate will vary somewhat from conjugate to conjugate, patient to patient, and will depend upon factors such as the condition of the patient, the loading capacity of the polymer conjugate, and the route of delivery. As a general proposition, a dosage from about 0.5 to about 100 mg/kg body weight, preferably from about 1.0 to about 20 mg/kg, will have therapeutic efficacy. When administered conjointly with other pharmaceutically active agents, even less of the polymer conjugate may be therapeutically effective. Typical routes of delivery include buccally, subcutaneously, transdermally, intramuscularly, intravenously, orally, or by inhalation.

The polymer conjugate may be administered once or several times a day. The duration of the treatment may be once per day for a period of from two to three weeks and may continue for a period of months or even years. The daily dose can be administered either by a single dose in the form of an individual dosage unit or several smaller dosage units or by multiple administration of subdivided dosages at certain intervals.

### V. EXAMPLES

The following examples are given to illustrate the invention, but should not be considered in limitation of the invention. For example, although PEG is used in the examples to illustrate the invention, other polymers that are useful in the practice of the invention are encompassed by the invention as discussed above.

All PEG reagents referred to in the appended examples are available from Shearwater Corporation of Huntsville, Ala. All ¹HNMR data was generated by a 300 or 400 MHz NMR spectrometer manufactured by Bruker.

### Example 1

### Preparation of di-PEG (20 kDa) Carboxylmethyl(CM) Conjugate of (I)

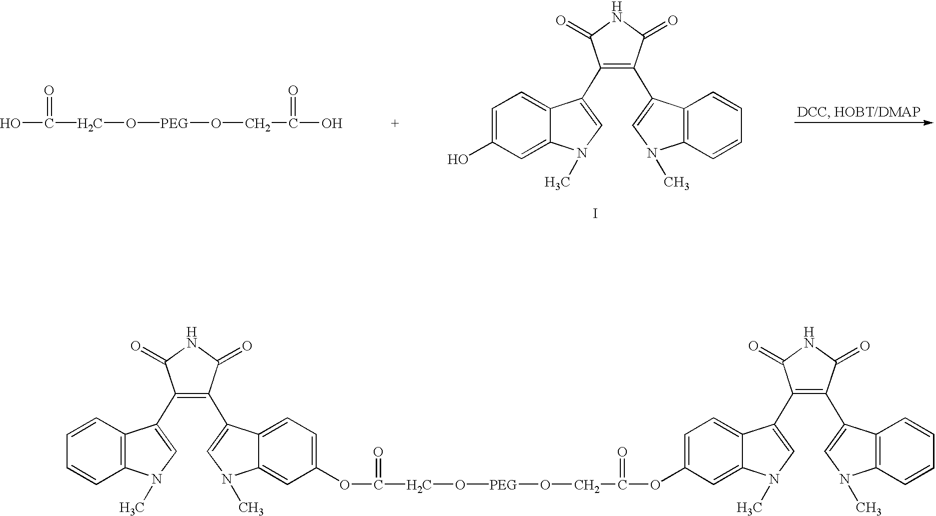

Compound I (46 mg), PEG-CM (20 kDa) (1 g), DCC 32 mg), HOBT (12 mg) and DMAP (17 mg) were dissolved in 25 ml of anhydrous methylene chloride. The solution was stirred overnight at room temperature under argon. The solvent was removed by rotary evaporation and the residue treated with 10 ml of toluene. The precipitate was removed by filtration, the solvent partially removed under vacuum, and the residual syrup added to 50 ml of ethyl ether. The precipitate was collected by filtration, washed with ether, and dried under vacuum. % substitution by nmr: >98%. ¹H NMR(DMSO-d₆): δ3.5 (br m, PEG), 4.37 (s, PEGOCH₂OCO—), 3.85 (s, N—CH₃), 3.80 (s, N—CH₃), 6.5-7.8 (M, aromatic H).

### Example 2

### Preparation of di-PEG (20 kDa) Propionate (PA) Conjugate of (I)

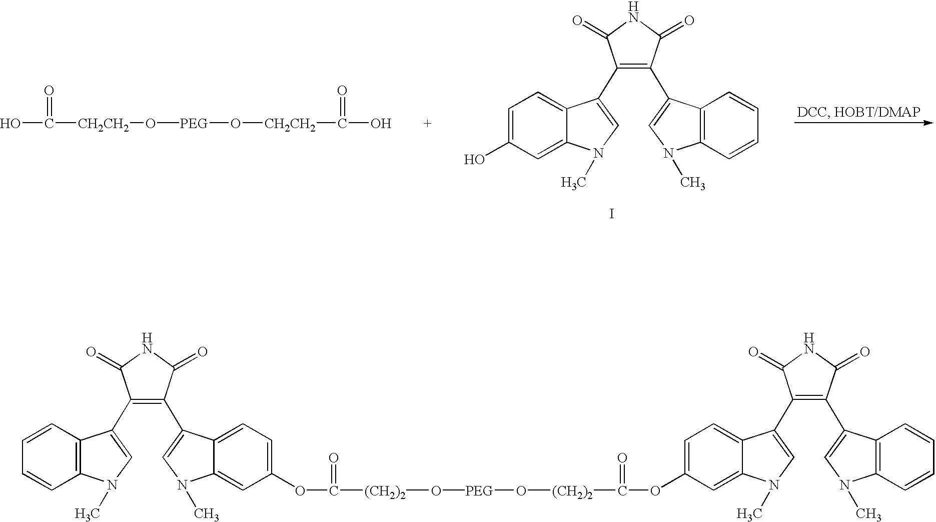

Compound I (49 mg), PEG-PA (20 kDa) (1.1 g), DCC (33 mg), HOBT (14.3 mg), and DMAP (18 mg) were dissolved in 25 ml of anhydrous methylene chloride. The solution was stirred overnight at room temperature under argon. The solvent was removed by rotary evaporation and the dried residue treated with 10 ml of toluene. The resulting precipitate was removed by filtration, the solvent partially removed under vacuum, and the residual syrup added to 50 ml of ethyl ether. The resulting precipitate was collected by filtration, washed sequentially with isopropyl alcohol and ether, and dried under vacuum. % substitution: >95%. ¹H NMR(DMSO-d₆): δ3.5 (br m, PEG), 2.76 (t, PEGOCH₂CH₂OCO—), 3.85 (s, N—CH₃), 3.80 (s, N—CH₃), 6.5-7.8 (M, aromatic H).

### Example 3

### Preparation of 4-arm PEG (10 kDa) Carboxylmethyl (CM) Conjugate of (I)

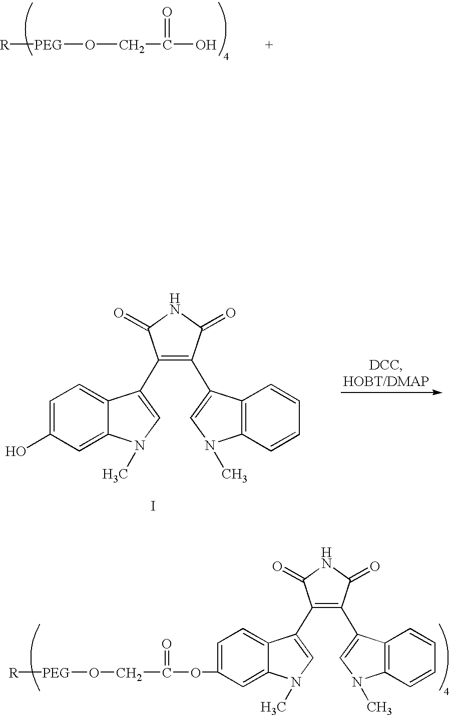

Compound I (23.5 mg), 4-arm PEG-CM (10 kDa) (150 mg), DCC (18 mg), HOBT (8.1 mg), and DMAP (8 mg) were dissolved in 25 ml of anhydrous methylene chloride. The solution was stirred overnight at room temperature under argon. The solvent was removed by rotary evaporation and the residue was treated with 10 ml of toluene. The resulting precipitate was removed by filtration, the solvent partially removed under vacuum and the residual syrup was to 100 ml of isopropyl alcohol/ethyl ether (50/50 ratio). The precipitate was collected by filtration, washed with ether, and dried under vacuum. Yield: 100 mg (58%). ¹H NMR(DMSO-d₆): δ3.5 (br m, PEG), 4.37 (s, PEGOCH₂OCO—), 3.85 (s, N—CH₃), 3.80 (s, N—CH₃), 6.5-7.8 (M, aromatic H).

### Example 4

### Preparation of di-PEG(20 kDa) Carboxylmethyl (CM) Conjugate of II

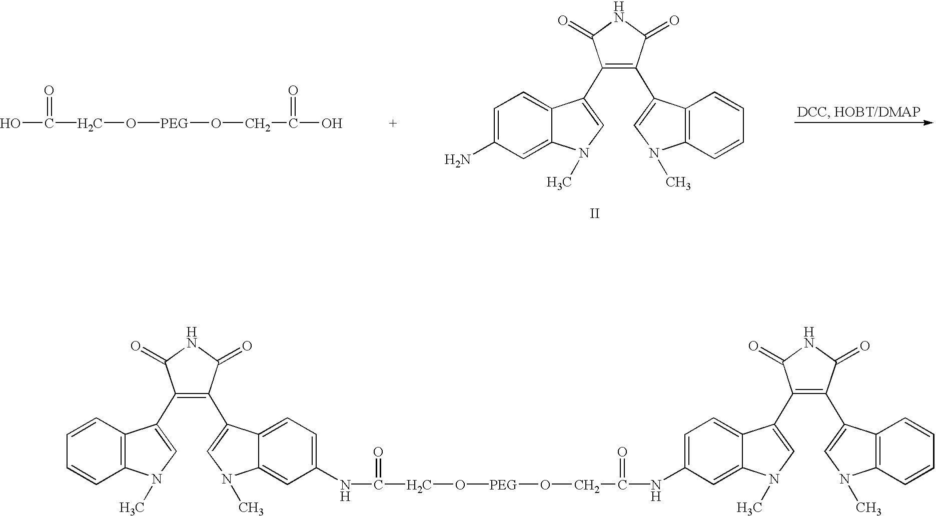

Compound II (46 mg), PEG-CM (20 kDa) (1 g), DCC (32 mg), HOBT (12 mg) and DMAP (17 mg) were dissolved in 25 ml of anhydrous methylene chloride. The solution was stirred overnight at room temperature under argon. The solvent was removed by rotary evaporation and the residue was treated with 10 ml of toluene. The precipitate was removed by filtration, the solvent was partially removed under vacuum and the syrup was added to 50 ml of ethyl ether. The precipitate was collected by filtration, washed with excess ether, and dried under vacuum. % substitution: >98%. ¹H NMR(DMSO-d₆): δ3.5 (br m, PEG), 4.02 (s, PEGOCH₂OCNH—), 3.85 (s, N—CH₃), 3.79 (s, N—CH₃), 6.5-7.8 (M, aromatic H), 9.43 (s, —CONH).

### Example 5

### Preparation of di-PEG (20 kDA)-CM-GA Conjugate of II

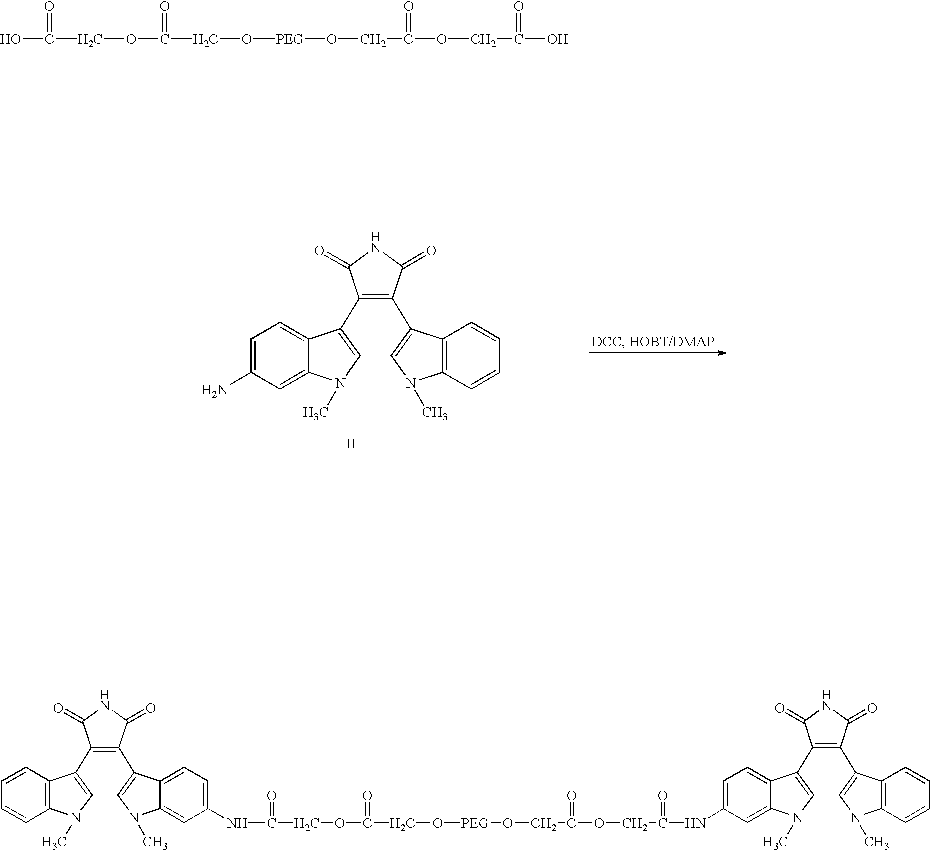

Compound II (20 mg, PEG-CM-GA 20 kDa (530 mg), DCC (17 mg), HOBT (7.2 mg) and DMAP (9 mg) were dissolved in 12 ml of anhydrous methylene chloride. The solution was stirred overnight at room temperature under argon. The solvent was removed by rotary evaporation and the residue was treated with 10 ml of toluene. The precipitate was removed by filtration, the solvent was partially removed under vacuum and the syrup was added to 50 ml of ethyl ether. The precipitate was collected by filtration, washed with ether, and dried under vacuum. % substitution: >99%. ¹H NMR(DMSO-d₆): δ3.5 (br m, PEG), 4.25 (s, PEGOCH₂COOCH₂OCNH—), 4.67 (s, PEGOCH₂COOCH₂OCNH—), 3.86 (s, N—CH₃), 3.79 (s, N—CH₃), 6.5-7.9 (M, aromatic H), 9.96 (s, —CONH).

### Example 6

### Preparation of mPEG (5 kDa)-PA-GA Conjugate of III

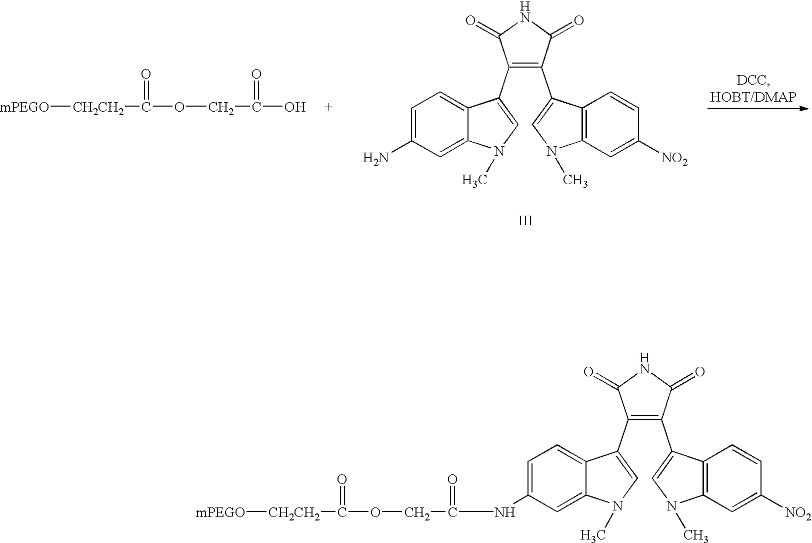

Compound III (25 mg), mPEG (5 kDa)-PA-GA (275 mg), DCC (16 mg), HOBT (7.8 mg) and DMAP (7.5 mg) were dissolved in 12 ml of methylene chloride. The solution was stirred overnight at room temperature under argon. The solvent was removed by rotary evaporation and the residue was treated with 10 ml of toluene. The precipitate was removed by filtration, the solvent was partially removed under vacuum and the syrup was added to 50 ml of ethyl ether. The precipitate was collected by filtration, washed with ether, and dried under vacuum. % substitution: >90%. ¹H NMR(DMSO-d₆): δ3.5 (br m, PEG), 2.65 (t, PEGOCH₂CH₂COOCH₂OCNH—), 4.61 (s, PEGOCH₂CH₂COOCH₂OCNH—), 4.00 (s, N—CH₃), 3.83 (s, N—CH₃), 6.5-8.5 (M, aromatic H), 9.91 (s, —CONH).

### Example 7

### Preparation of mPEG (5 kDa)-PA Conjugate of IV

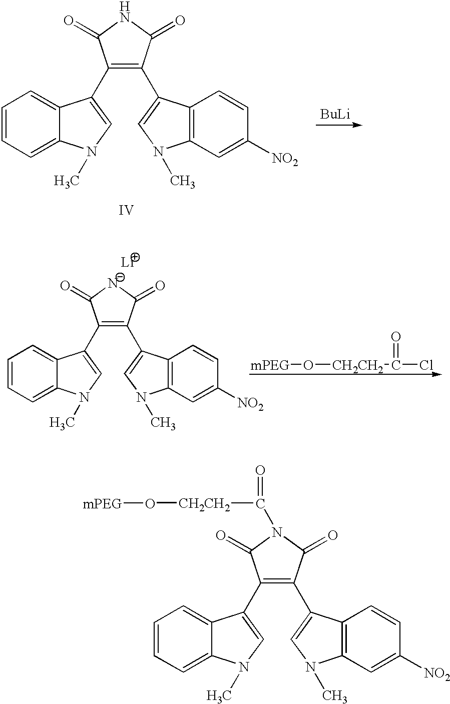

mPEG (5 kDa)-PA (250 mg) was dissolved in 5 ml of methylene chloride. To this solution was added thionyl chloride (0.4 ml, 2M) in dichloromethane. The solution was stirred overnight and the solvent was removed under vacuum. The residue was dissolved in dioxane (2 ml) and placed under argon.

Compound IV (23 mg) was dissolved in THF (5 ml). The solution was cooled under argon to 0° C. and to it was added dropwise 28 μl of butyllithium (2M in hexane). The solution was stirred at 0° C. for 10 minutes and then added to the solution of mPEG-PA chloride (previous step). The solution was stirred at room temperature under argon for 5 hours. The solvent was removed by rotary evaporation and the residual syrup was added to 50 ml of ethyl ether. The resulting precipitate was collected by filtration, washed with ether, and dried under vacuum. % substitution: >60%. ¹H NMR(DMSO-d₆): δ3.5 (br in, PEG), 4.00 (s, N—CH₃), 3.91 (s, N—CH₃), 6.5-8.5 (M, aromatic H).

### Example 8

### Preparation of MPEG (5 kDa)-CM Conjugate of IV

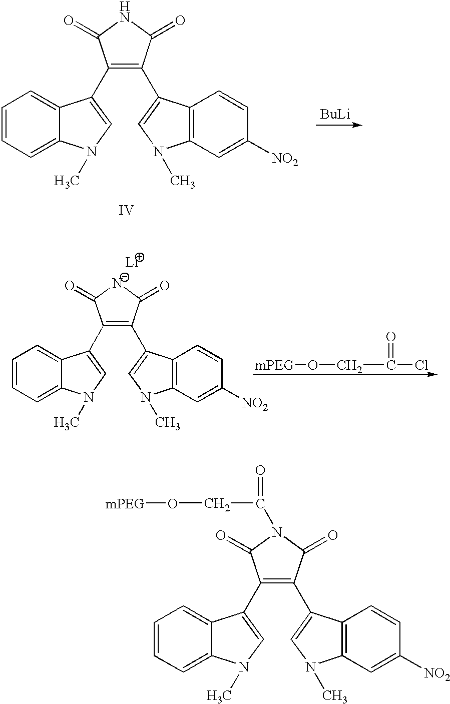

mPEG (5 kDa)-CM (250 mg) was dissolved in methylene chloride (5 ml). To this solution was added thionyl chloride (0.4 ml) (2M, in dichloromethane). The solution was stirred overnight and the solvent was removed under vacuum. The residue was dissolved in dioxane (2 ml) and placed under argon.

Compound IV (25 mg) was dissolved in THF (5 ml). The solution was cooled under argon to 0° C. and 28.8 μl of butyllithium (2M in hexane) was added to it dropwise. The solution was stirred at 0° C. for 10 minutes and then added to the solution of mPEG-PA chloride (previous step). The resulting solution was stirred at room temperature under argon for 5 hours. The solvent was removed by rotary evaporation and the residual syrup was added to 50 ml of ethyl ether. The resulting precipitate was collected by filtration, washed with ether, and dried under vacuum. % substitution: >84%. ¹H NMR(DMSO-d₆): δ3.5 (br m, PEG), 4.70 (s, mPEGOCH₂CON—), 4.00 (s, N—CH₃), 3.92 (s, N—CH₃), 6.5-8.5 (M, aromatic H).

### Example 9

### Hydrolysis Half-lives of the Ester Linkage of PEG Conjugates of PKC Inhibitor Compounds

The conjugates were dissolved with a PEG internal standard in phosphate buffer (pH 7.2), and incubated at 37° C. or at room temperature (23 ° C.). At timed intervals, solutions were analyzed by HPLC using an Ultrahydrogel 250 column (Waters). The hydrolysis half-lives of the ester linkages are listed in Table 1 below.

TABLE 1
Hydrolysis Half-lives of PEG Prodrugs of PKC Inhibitor Compounds

| | 37° C. | 23° C. |
|---|---|---|
| PEG-CM conjugate of | 71 hrs | 8.5 hrs |
| Example 1 | | |
| PEG-PA conjugate of | 50 days | 7 days |
| Example 2 | | |
| PEG-CM-GA conjugate of | 3.2 days | 14 hrs |
| Example 5 | | |

As the above data suggests, the ester linkages within the prodrug conjugates of Examples 1, 2, and 5 hydrolyze over time to release the PKC inhibitor molecule.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A polymer conjugate comprising a water soluble and non-peptidic polymer covalently attached to a protein kinase C inhibitor.

2. The polymer conjugate of claim 1, wherein the water soluble and non-peptidic polymer is covalently attached via a hydrolytically degradable linkage to the protein kinase C inhibitor.

3. The polymer conjugate of claim 2, wherein the hydrolytically degradable linkage is selected from the group consisting of carboxylate ester, phosphate ester, anhydride, acetal, ketal, acyloxyalkyl ether, imine, orthoester, and oligonucleotide.

4. The polymer conjugate of claim 1, wherein the polymer is selected from the group consisting of poly(alkylene glycol), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), and copolymers, terpolymers, and mixtures thereof.

5. The polymer conjugate of claim 1, wherein the polymer is poly(ethylene glycol).

6. The polymer conjugate of claim 1, wherein the protein kinase C inhibitor selectively inhibits the alpha, beta, or gamma protein kinase C isozyme.

7. The polymer conjugate of claim 1, wherein the protein kinase C inhibitor is a indolylmaleimide or indazolyl-substituted pyrroline molecule.

8. The polymer conjugate of claim 1, wherein the protein kinase C inhibitor is a indolylmaleimide molecule and the polymer is attached to a carbon atom of an indole ring or the nitrogen atom of the maleimide ring.

9. The polymer conjugate of claim 8, wherein the indolylmaleimide molecule has the structure: 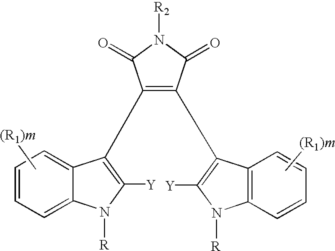
wherein:
each R is independently selected from the group consisting of alkyl, substituted alkyl, cycloalkyl, substituted cycloalkyl, alkenyl, alkynyl, aryl, substituted aryl, heteroaryl, substituted heteroaryl, heterocycle, and substituted heterocycle, or both R groups together form -T-W-J-, wherein W is —O—, —S—, —SO—, —SO₂—, —CO—, C2-C6alkylene, substituted C2-C6alkylene, C2-C6alkenylene, -arylene-, -arylene-alkylene-O—, -heterocycle-, -heterocycle-alkylene-O—, -cycloalkyl-alkylene-O—, —NR₃—, —NOR₃—, —CONH—, or —NHCO—, where R₃ is hydrogen, alkyl, substituted alkyl, —C(O)O-alkyl, aminocarbonyl, amidino, alkylsulphinyl, aminosulphonyl, or alkylsulphonyl, and T and J are independently C1-C6alkylene or substituted C1-C6alkylene, or T, W, and J together form —C2-C6alkylene-AA-, where AA is an amino acid residue;
each R₁ is independently selected from the group consisting of halo, hydroxy, alkyl, substituted alkyl, alkoxy, substituted alkoxy, aryloxy, substituted aryloxy, nitro, thiol, amino, substituted amino, alkylsulphinyl, alkylsulphonyl, and alkylthio;
m is 0-4;
R₂ is selected from the group consisting of hydrogen, halo, hydroxy, alkyl, substituted alkyl, alkoxy, substituted alkoxy, amino, substituted amino, and alkylcarbonyl; and
each Y is independently selected from the group consisting of hydrogen, alkyl, substituted alkyl, alkylthio, and alkylsulphinyl, or Y together with R, form a fused C3-C8 heterocyclic ring, optionally substituted with one or more alkyl, substituted alkyl, or amino groups.

10. The polymer conjugate of claim 9, wherein Y and R₂ are hydrogen and each R is independently selected from the group consisting of hydrogen, alkyl, haloalkyl, hydroxyalkyl, alkoxyalkyl, alkylamino, alkylaminoalkyl, dialkylaminoalkyl, trialkylaminoalkyl, aminoalkylaminoalkyl, azidoalkyl, acylaminoalkyl, acylthioalkyl, alkylsulphonylaminoalkyl, arylsulphonylaminoalkyl, mercaptoalkyl, alkylthioalkyl, alkylsuphinylalkyl, alkylsulphonylalkyl, alkylsulphonyloxyalkyl, alkylcarbonyloxyalkyl, cyanoalkyl, amidinoalkyl, isothiocyanatoalkyl, glucopyranosyl, carboxyalkyl, alkoxycarbonylalkyl, aminocarbonylalkyl, hydroxyalkylthioalkyl, mercaptoalkylthioalkyl, arylthioalkyl, carboxyalkylthioalkyl, alkyl-S(C═NH)NH₂, and alkyl-NC(═NNO₂)NH₂.

11. The polymer conjugate of claim 1, having the structure:
Z-POLY-X—I_{PKC}
wherein:
Z is a capping group or a functional group;
POLY is a water soluble and non-peptidic polymer;
X is a linkage; and
I_{PKC} is a protein kinase C inhibitor.

12. The polymer conjugate of claim 11, wherein POLY is poly(ethylene glycol).

13. The polymer conjugate of claim 11, wherein X is selected from the group consisting of —CONH—, —C(O)—, —O—(CH₂)ₙ—C(O)—O— where n is 1-10, —O—(CH₂)ₙ—C(O)—NH— wherein n is 1-10, —C(O)—O—(CH₂)ₙ—C(O)—NH— where n is 1-10, and —O—CH₂—C(O)O—CH₂—C(O)—NH—.

14. The polymer conjugate of claim 11, having the structure: 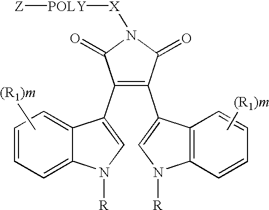
wherein:
each R is independently selected from the group consisting of alkyl, substituted alkyl, cycloalkyl, substituted cycloalkyl, alkenyl, alkynyl, aryl, substituted aryl, heteroaryl, substituted heteroaryl, heterocycle, and substituted heterocycle, or both R groups together form -T-W-J-, wherein W is —O—, —S—, —SO—, —SO₂—, —CO—, C2-C6alkylene, substituted C2-C6alkylene, C2-C6alkenylene, -arylene-, -arylene-alkylene-O—, -heterocycle-, -heterocycle-alkylene-O—, -cycloalkyl-alkylene-O—, —NR₃—, —NOR₃—, —CONH—, or —NHCO—, where R₃ is hydrogen, alkyl, substituted alkyl, —C(O)O-alkyl, aminocarbonyl, amidino, alkylsulphinyl, aminosulphonyl, or alkylsulphonyl, and T and J are independently C1-C6alkylene or substituted C1-C6alkylene, or T, W, and J together form —C2-C6alkylene-AA-, where AA is an amino acid residue;
each R₁ is independently selected from the group consisting of halo, hydroxy, alkyl, substituted alkyl, alkoxy, substituted alkoxy, aryloxy, substituted aryloxy, nitro, thiol, amino, substituted amino, alkylsulphinyl, alkylsulphonyl, and alkylthio; and
m is 0-4.

15. The polymer conjugate of claim 11, having the structure: 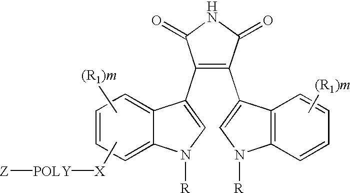
wherein:
each R is independently selected from the group consisting of alkyl, substituted alkyl, cycloalkyl, substituted cycloalkyl, alkenyl, alkynyl, aryl, substituted aryl, heteroaryl, substituted heteroaryl, heterocycle, and substituted heterocycle, or both R groups together form -T-W-J-, wherein W is —O—, —S—, —SO—, —SO₂—, —CO—, C2-C6alkylene, substituted C2-C6alkylene, C2-C6alkenylene, -arylene-, -arylene-alkylene-O—, -heterocycle-, -heterocycle-alkylene-O—, -cycloalkyl-alkylene-O—, —NR₃—, —NOR₃—, —CONH—, or —NHCO—, where R₃ is hydrogen, alkyl, substituted alkyl, —C(O)O-alkyl, aminocarbonyl, amidino, alkylsulphinyl, aminosulphonyl, or alkylsulphonyl, and T and J are independently C1-C6alkylene or substituted C1-C6alkylene, or T, W, and J together form —C2-C6alkylene-AA-, where AA is an amino acid residue;
each R₁ is independently selected from the group consisting of halo, hydroxy, alkyl, substituted alkyl, alkoxy, substituted alkoxy, aryloxy, substituted aryloxy, nitro, thiol, amino, substituted amino, alkylsulphinyl, alkylsulphonyl, and alkylthio; and
m is 0-4.

16. The polymer conjugate of claim 1, having the structure: 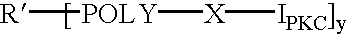
wherein:
each POLY is a water soluble and non-peptidic polymer;
R′ is a central core molecule;
y is from about 3 to about 100;
X is a linkage; and
I_{PKC} is a protein kinase C inhibitor.

17. The polymer conjugate of claim 16, wherein R′ is a residue of a central core molecule selected from the group consisting of glycerol, glycerol oligomers, pentaerythritol, sorbitol, and lysine.

18. The polymer conjugate of claim 16, wherein each POLY is poly(ethylene glycol).

19. The polymer conjugate of claim 1, wherein the polymer is linear or branched.

20. A pharmaceutical composition, comprising:
a polymer conjugate comprising a water soluble and non-peptidic polymer covalently attached to a protein kinase C inhibitor, and
a pharmaceutically acceptable carrier.

21. The pharmaceutical composition of claim 20, wherein the polymer is selected from the group consisting of poly(alkylene glycol), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazoline, poly(N-acryloylmorpholine), poly(acrylic acid), carboxymethyl cellulose, hyaluronic acid, hydroxypropylmethyl cellulose and copolymers, terpolymers, and mixtures thereof.

22. The pharmaceutical composition of claim 20, wherein the polymer is poly(ethylene glycol).

23. The pharmaceutical composition of claim 20, wherein the protein kinase C inhibitor selectively inhibits the alpha, beta, or gamma protein kinase C isozyme.

24. The pharmaceutical composition of claim 20, wherein the protein kinase C inhibitor is a indolylmaleimide or indazolyl-substituted pyrroline molecule.

25. The pharmaceutical composition of claim 20, wherein the protein kinase C inhibitor is a indolylmaleimide molecule and the polymer is attached to a carbon atom of either indole ring or the nitrogen atom of the maleimide ring.
